SEQUENCE LISTING (12 sequences)

SEQ ID NO: 1 - protein, 652 aa, Homo sapiens [sequence omitted]

SEQ ID NO: 2 - DNA, 3350 bp, Homo sapiens
  misc_feature: Incyte ID No: 429905
  unsure 3293: a, t, c, g, or other
gcgtgggatacccgtaccacagaaatgcagggaccattgcttcttccaggcctctgctttctgctgagcctctttggagctgtgactcagaaaaccaaaacttcctgtgctaagtgccccccaaatgcttcctgtgtcaataacactcactgcacctgcaaccatggatatacttctggatctgggcagaaactattcacattccccttggagacatgtaacgacattaatgaatgtacaccaccctatagtgtatattgtggatttaacgctgtatgttacaatgtcgaaggaagtttctactgtcaatgtgtcccaggatatagactgcattctgggaatgaacaattcagtaattccaatgagaacacctgtcaggacaccacctcctcaaagacaacccagggcaggaaagagctgcaaaagattgtggacaaatttgagtcacttctcaccaatcagactttatggagaacagaagggagacaagaaatctcatccacagctaccactattctccgggatgtggaatcgaaagttctagaaactgccttgaaagatccagaacaaaaagtcctgaaaatccaaaacgatagtgtagctattgaaactcaagcgattacagacaattgctctgaagaaagaaagacattcaacttgaacgtccaaatgaactcaatggacatccgttgcagtgacatcatccagggagacacacaaggtcccagtgtcattgcctttatctcatattcttctcttggaaacatcataaatgcaactttttttgaagagatggataagaaagatcaagtgtatctgaactctcaggttgtgagtgctgctattggacccaaaaggaacgtgtctctctccaagtctgtgacgctgactttccagcacgtgaagatgacccccagtaccaaaaaggtcttctgtgtctactggaagagcacagggcagggcagccagtggtccagggatggctgcttcctgatacacgtgaacaagagtcacaccatgtgtaattgcagtcacctgtccagcttcgctgtcctgatggccctgaccagccaggaggaggatcccgtgctgactgtcatcacctacgtggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggggattgatcgaactgaacccaaggtgctgtgctccatcatcgccggtgctttgcactatctctacctggccgccttcacctggatgctgctggagggtgtgcacctcttcctcactgcacggaacctgacagtggtcaactactcaagcatcaatagactcatgaagtggatcatgttcccagtcggctatggcgttcccgctgtgactgtggccatttctgcagcctcctggcctcacctttatggaactgctgatcgatgctggctccacctggaccagggattcatgtggagtttccttggcccagtctgtgccattttctctgcgaatttagtattgtttatcttggtcttttggattttgaaaagaaaactttcctccctcaatagtgaagtgtcaaccatccagaacacaaggatgctggctttcaaagcaacagctcagctcttcatcctgggctgcacatggtgtctgggcttgctacaggtgggtccagctgcccaggtcatggcctacctcttcaccatcatcaacagcctccaaggcttcttcatcttcttggtctactgcctcctcagccagcaggtccagaaacaatatcaaaagtggtttagagagatcgtaaaatcaaaatctgagtctgagacatacacactttccagcaagatgggtcctgactcaaaacccagtgagggggatgtttttccaggacaagtgaagagaaaatattaaaactagaatattcaactccatatggaaaatcatatccatggatctctttggcattatgaagaatgaagctaaggaaaagggaattcattaaacatatcatccttggagaggaagtaatcaacctttacttcccaaactgtttgttctccacaataggctctcaacaaatgtgtggtaaattgcatttctcttcactatggtgtattcagtcaatgcttgtccctggaaacccaaagcatgaccactgcaaatatttccttgactttttgtaaatgaagaggtccttttcctcaagttcttagtcccactcatcctaaacttgctctttttttaagacagagtttcactctgtcacccaggctggagtgtagtggcatgatcgtagctcactgcagcctcaaactccagagctcaactggttctccagcctcagcttcccaaagtgctgggattacaggcatgagccactgcacctggccataaacttgctctttaaactcactcattccctcaaaccatcagcttcctactggctttacttccttgctagatacaggctaatttttttttttttttttttttttttttgagatggagtttcgctcttgttgcccaggctggagtgcaacggcgtgagtgcaacctctgcctcccgggttcaagcgattcttctgcctcagcctcccaagtagctggcgttacaggtatggaccaccatgtccggctaattttgtatttttagtagagacagggtttctccatgttggtcaggctggtctcgaactcccagcctcaggtgatccacctgacttggcctcccaagagtgctgggattacaggcatgagccaccgtgcccagcccaggctaacttattttcttctgagactgagtctcactactgtcacccaggctggagtgcagtggtgagatctaggctcactgcaacctctacctcctgggttcaagcaattctcctgccttagcctcccgatagctgggactacaagcacatgccgccatgcccagctaattttgtatttttagtggagacaaggtttcaccatgttggccaggctgatctcaaactcctgacctcaagcagcgatccacctgccggggcctcccaaagtgctgggattacagacacaagccatcgcgcctgatgagagattttaagtgttctcaccacaaaaaaaaagaaaaaaaagttatatgaggtaatcgtatattaattagcttgacttagtcattccacgatgtagatatatttcaaaacatcctgttgtacatgataaatatatatattttngtctatataaaacaaataaataaataaatgtttaaagtgtaaaaaaaaaaaaaaaag

SEQ ID NO: 3 - DNA, 153 bp, Homo sapiens
  misc_feature: Incyte ID No: 429905H1
  unsure 4, 8, 16, 19, 65: a, t, c, g, or other
ggtngctngcgtgggntanccgtaccacaggaatccagggaccattggttttttcagggctctgntttctgctgaggctctttggagctgtgactcagaaaaccaaaacttcctgtgctaagtgccccccaaatgcttcctgtgtcaataaca

SEQ ID NO: 4 - DNA, 320 bp, Homo sapiens
  misc_feature: Incyte ID No: 1837630F6
cggcacagtagagagcttccagggctggctggcgtgggatacccgtaccacagaactgcagggaccattgcttcttccaggcctctgctttctgctgagcctctttggagctgtgactcagaaaaccaaaacttcctgtgctaagtgccccccaaatgcttcctgtgtcaataacactcactgcacctgcaaccatggatatacttctggatctgggcagaaactattcacattccccttggagacatgtaacgacattaatgaatgtacaccaccctatagtgtatattgtggatttaacgctgtgtgttacaatgtcg

SEQ ID NO: 5 - DNA, 624 bp, Homo sapiens
  misc_feature: Incyte ID No: 8009216H1
ggggaggaaaggtggttggcttacggcacagtagagagcttccaggctggctggcgtgggatacccgtaccacagaaatgcaggaccattgcttcttccaggcctctgctttctgctgagcctctttggagctgtgactcagaaaaccaaaacttcctgtgctaagtgccccccaaatgcttcctgtgtcaataacactcactgcacctgcaaccatggatatacttctggatctgggcagaaactattcacattccccttggagacatgtaacgacattaatgaatgtacaccaccctatagtgtatattgtggatttaacgctgtgtgttacaatgtcgaaggaagtttctactgtcaatgtgtcccaggatatagactgcattctgggaatgaacaattcagtaattccaatgagaacacctgtcaggacaccacctcctcaaagacaacccagggcaggaaagagctgcaaaagattgtggacaaatttgagtcacttctcaccaatcagactttatggagaacagaatggagacaagaaatctcatccacaggtaccactattctccgggatgtggaatcgaaagttctagaaactggcttgaaagatccagaacaaaa

SEQ ID NO: 6 - DNA, 554 bp, Homo sapiens
  misc_feature: Incyte ID No: 1837630T6
caaacagcttgggaagtaaaggttgattacttcctctccaaggatgatatgtttaatgaattcccttttccttagcttcattcttcataatgccaaagagatccatggatatgattttccatatggagttgaatattctagttttaatatgttctcttcacttgtcctggaaaaacatccccctcactgggttttgagtcaggacccatcttgctggaaagtgtgtatgtctcagactcagattttgattttacgatctcgctaaaccacttttgatattgtttctggacctgctggctgaggaggcagtagaccaagaagatgaagaagccttggaggctgttgatgatggtgaagaggtaggccatgacctgggcagctggacccacctgtagcaagcccagacaccatgtgcagcccaggatgaagagctgagctgttgctttgaaagccagcatccttgtgttctggatggttgacacttcactattgagggaggaaagttttcttttcaaaatccaaaagaccaagataaacaatactaaattcgcaga

SEQ ID NO: 7 - DNA, 514 bp, Homo sapiens
  misc_feature: Incyte ID No: 2438817F7
gggagacacacaaggtcccaggccattgcctttatctcatattcttctctggaaacatcataaatgcaactttttttgaagagatggataagaaagatcaagtgtatctgaactctcaggtgtgagtgctgctatggacccaaaaggaacgtgtctctctccaagtctgtgacgctgactttccagcacgtgaagatgacccccagtaccaaaaaggtcttctgtgtctactggaagagcacagggcagggcagccagtggtccaggatggctgcttcctgatacacgtgaacaagagtcacaccatgtgtaattgcagtcacctgtccagcttcgctgtcctgatggccctgaccagccaggaggaggatcccgtgctgactgtcatcacctacgtggggctgagcgtctctctgctgtgcctcctcctggcgggcctcacttttctcctgtgtaaagccatccagaacaccagcacctcactgcatctgcagctctcgctctggctcttcct

SEQ ID NO: 8 - DNA, 445 bp, Homo sapiens
  misc_feature: Incyte ID No: 320551R1
  unsure 6, 17, 76, 105: a, t, c, g, or other
gtccanctgcccaggtncatggcctacctcttcaccatgcatcaacagcctccaaggcttcttcatcttcttggtnctactgcctcctcagccagcaggtccagnaaacaatatcaaaagtggtttagaggagatcgttaaaatcaaaatctgagtctgaggacatacacactttccagcaagatgggtcctgactcaaaacccagtgtagggggatgtttttccaggacaagtgaagaggaaaatattaaaactaggaatattcaactccatatggaaaatcatatccatggatctctttggcattatgaagaatgaagctaaggaaaagggaattcattaaacatatcatccttggagaggaagtaatcaacctttacttcccaagctgtttgttctccacaataggctctcaacaaatgtgtggtaaattgcatttctcttc

SEQ ID NO: 9 - DNA, 419 bp, Homo sapiens
  misc_feature: Incyte ID No: 524802R6
gaaatctcatccacagctagcactattctccgggatgtggaatcgaaagttctagaaactgccttgaaagatccagaacaaaaagtcctgaaaatccaaaacgatagtgtagctattgaaactcaagcgattacagacaattgctctgaagaaagaaagacattcaacttgaacgtccaaatgaactcaatggacatccgttgcagtgacatcatccagggagacacacaaggtcccagtgccattgcctttatctcatattcttctcttggaaacatcataaatgcaactttttttgaagagatggataagaaagatcaagtgtatctgaactctcaggttgtgagtgctgctattggacccaaaaggaacgtgtctctctccaagtctgtgacgctgactttccagcacgtgaagat

SEQ ID NO: 10 - DNA, 598 bp, Homo sapiens
  misc_feature: Incyte ID No: 7749732H1
cagcacctcactgcatctgcagctctcgctctgcctcttcctggcccacctcctcttcctcgtggggattgatcgaactgaacccaaggtgctgtgctccatcatcgccggtgctttgcactatctctacctggccgccttcacctggatgctgctggagggtgtgcacctcttcctcactgcacggaacctgacagtggtcaactactcaagcatcaatagactcatgaagtggatcatgttcccagtcggctatggcgttcccgctgtgactgtggccatttctgcagcctcctggcctcacctttatggaactgctgatcgatgctggctccacctggaccagggattcatgtggagtttccttggcccagtctgtgccattttctctgcgaatttagtattgtttatcttggtcttttggattttgaaaagaaaactttcctccctcaatagtgaagtgtcaaccatccagaacacaaggatgctggctttcaaagcaacagctcagctcttcatcctgggctgcacatggtgtctgggcttgctacaggtgggtccagctgcccaggtcatggcctacctcttcaccatcatc

SEQ ID NO: 11 - protein, 886 aa, Homo sapiens
  ref <308>: DATABASE ACCESSION NUMBER: Genbank ID No: g784994
MRGFNLLLFWGCCVMHSWEGHIRPTRKPNTKGNNCRDSTLCPAYATCTNTVDSYYCTCKQGFLSSNGQNHFKDPGVRCKDIDECSQSPQPCGPNSSCKNLSGRYKCSCLDGFSSPTGNDWVPGKPGNFSCTDINECLTSRVCPEHSDCVNSMGSYSCSCQVGFISRNSTCEDVNECADPRACPEHATCNNTVGNYSCFCNPGFESSSGHLSCQGLKASCEDIDECTEMCPINSTCTNTPGSYFCTCHPGFAPSSGQLNFTDQGVECRDIDECRQDPSTCGPNSICTNALGSYSCGCIVGFHPNPEGSQKDGNFSCQRVLFKCKEDVIPDNKQIQQCQEGTAVKPAYVSFCAQINNIFSVLDKVCENKTTVVSLKNTTESFVPVLKQISMWTKFTKEETSSLATVFLESVESMTLASFWKPSANVTPAVRAEYLDIESKVINKECSEENVTLDLVAKGDKMKIGCSTIEESESTETTGVAFVSFVGMESVLNERFFQDHQAPLTTSEIKLKMNSRVVGGIMTGEKKDGFSDPIIYTLENVQPKQKFERPICVSWSTDVKGGRWTSFGCVILEASETYTICSCNQMANLAVIMASGELTMDFSLYIISHVGIIISLVCLVLAIATFLLCRSIRNHNTYLHLHLCVCLLLAKTLFLAGIHKTDNKTGCAIIAGFLHYLFLACFFWMLVEAVILFLMVRNLKVVNYFSSRNIKMLHICAFGYGLPMLVVVISASVQPQGYGMHNRCWLNTETGFIWSFLGPVCTVIVINSLLLTWTLWILRQRLSSVNAEVSTLKDTRLLTFKAFAQLFILGCSWVLGIFQIGPVAGVMAYLFTIINSLQGAFIFLIHCLLNGQVREEYKRWITGKTKPSSQSQTSRILLSSMPSASKTG

SEQ ID NO: 12 - protein, 344 aa, Homo sapiens
  ref <308>: DATABASE ACCESSION NUMBER: Genbank ID No: g2935597
KVLCVFWEHGQNGCGHWATTGCSTIGTRDTSTICRCTHLSSFAVLMAHYDVQEDPVLTVITYMGLSVSLLCLLLAALTFLLCKAIQNTSTSLHLQLSLCLFLAHLLFLVAIDQTGHKVLCSIIAGTLHYLYLATFTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

(19) United States
(12) Patent Application Publication
(10) Pub. No.: US 2002/0146767 A1
(43) Pub. Date: Oct. 10, 2002

(54) Human EMR1-like G protein-coupled receptor

(76) Inventors: Hong Xu, Mystic, CT (US); Victoria L. Cohan, East Lyme, CT (US); Susan G. Stuart, Montara, CA (US)

Correspondence Address: INCYTE GENOMICS, INC., LEGAL DEPARTMENT, 3160 PORTER DRIVE, PALO ALTO, CA, 94304, US

(21) Appl. No.: 09/992,647

(22) Filed: Nov. 13, 2001

Related U.S. Application Data
(63) Continuation-in-part of application No. 09/368,394, filed on Aug. 4, 1999, now abandoned.
(63) Continuation-in-part of application No. 09/110,116, filed on Jul. 2, 1998, now Pat. No. 6,013,479.

(51) Int. Cl. G01N 33/53; C07H 21/04; C12P 21/02; C12N 5/06; C07K 14/705; C12P 21/04; C12M 1/34

(52) U.S. Cl. 435/69.1; 435/320.1; 435/325; 530/350; 536/23.2; 435/7.1; 435/287.2; 435/70.21

(57) ABSTRACT

The invention provides a cDNA which encodes a human Emr1-like G protein-coupled receptor. It also provides for the use of the cDNA, protein, and antibody in the diagnosis, prognosis, treatment and evaluation of therapies for respiratory, inflammatory, and immunological disorders. The invention further provides vectors and host cells for the production of the protein and transgenic model systems.

This application is a continuation-in-part of U.S. Ser. No. 09/368,394, filed Aug. 4, 1999; and U.S. Ser. No. 09/110,116, filed Jul. 2, 1998, issued as U.S. Pat. No. 6,013,479, on Jan. 11, 2000.

### FIELD OF THE INVENTION

This invention relates to a human Emr1-like G protein-coupled receptor, its encoding cDNA, and an antibody which binds the protein which can be used in the diagnosis, prognosis, treatment and evaluation of therapies for respiratory, inflammatory, and immunological disorders.

### BACKGROUND OF THE INVENTION

Inflammation is a molecular, cellular, and tissue program during which foreign substances and pathogens are destroyed, and injured tissue is repaired through a variety of biochemical, biophysical, and cellular mechanisms. Among the first indications that inflammation is occurring is the production of leukocytes. Microscopic analysis of a peripheral blood sample shows an increase in the number of eosinophils and production of two main classes of leukocytes that sustain the inflammatory processes, granulocytes and monocytes/macrophages. Macrophages form an important part of the mammalian host defense system and function in both normal and pathological processes.

Signal transduction is the general process by which cells respond to extracellular signals. Extracellular signals are mediated through a biochemical cascade that begins with the binding of a signal molecule, e.g., a hormone, neurotransmitter, or growth factor, to a cell membrane receptor and ends with the activation of an intracellular target molecule. This process of signal transduction regulates all types of cell functions including cell proliferation, differentiation, and gene transcription.

GTP binding protein (G protein) signaling is one of the important biochenical pathways of signal transduction. G protein-coupled receptors (GPCR) are a superfamily of integral membrane proteins which transduce extracellular signals. GPCRs include receptors for biogenic amines; for lipid mediators of inflammation, peptide hormones, and sensory signal mediators. The GPCR becomes activated when the receptor binds its extracellular ligand. Conformational changes in the GPCR which result from the ligand-receptor interaction affect the binding affinity of a G protein to the GPCR intracellular domains. This enables GTP to bind with enhanced affinity to the G protein. Activation of the G protein by GTP leads to the interaction of the G protein a subunit with adenylate cyclase or other second messenger molecule generator. This interaction regulates the activity of adenylate cyclase and hence production of a second messenger molecule, cAMP. cAMP regulates phosphorylation and activation of other intracellular proteins. Alternatively, cellular levels of other second messenger molecules, such as, for example, cGMP or eicosinoids, may be upregulated or downregulated by the activity of GPCRs. The G protein a subunit is deactivated by hydrolysis of the GTP by GTPase and the βγ and α subunits reassociate. The heteromeric G protein then dissociates from the adenylate cyclase or other second messenger molecule generator. Activity of GPCR may also be regulated by phosphorylation of the intra- and extra-cellular domains or loops.

The structure of these highly-conserved receptors consists of seven hydrophobic transmembrane (serpentine) regions, cysteine disulfide bridges between the second and third extracellular loops, an extracellular N-terminus, and a cytoplasmic C-terminus. Three extracellular loops alternate with three intracellular loops to link the seven transmembrane regions. The most conserved parts of these proteins are the transmembrane regions and the first two cytoplasmic loops. A conserved, acidic-Arg-aromatic residue triplet present in the second cytoplasmic loop may interact with the G-proteins. The consensus pattern of the G-protein coupled receptors signature (PS00237; SWISSPROT) is characteristic of most proteins belonging to this superfamily (Watson and Arkinstall (1994) The G-protein Linked Receptor Facts Book, Academic Press, San Diego Calif., pp 2-6).

Examples of GPCRs implicated in inflammation and the immune response include the EGF module-containing, mucin-ike hormone receptor (Emr1; g784994, NCBI GenBank) and CD97β receptor proteins. These seven transmembrane hormone receptors exist as heterodimers in vivo and contain between three and seven potential calcium-binding EGF-like motifs (Baud et al. (1995) Genomics 26:334-344; Gray et al. (1996) J Immunol 157:5438-5447). In addition, an orphan Emr1-like GPCR (g2935597, NCBI GenBank) has recently been mapped to human chromosome 19. These GPCRs are members of the recently characterized EGF-TM7 receptors family.

Like other seven transmembrane receptors, the EGF-TM7 receptors are of extremely low abundance; they are very sparsely expressed. Small changes in the numbers of these receptors can lead to substantial changes for the cells in which they reside; their extreme sensitivity to input signals can initiate a large-scale coordinated response. Furthermore, “changes in receptor protein levels are usually mirrored by changes in receptor mRNA levels” (Zweiger (2001) Transducing the Genome, McGraw Hill, New York N.Y.).

The discovery of a new human Emr1-like G protein-coupled receptor, an encoding DNA, and an antibody which specifically binds the protein satisfies a long standing need in the art by providing molecules and compositions which can be used in the diagnosis, prognosis, treatment and evaluation of therapies for respiratory, inflammatory, and immunological disorders.

### SUMMARY OF THE INVENTION

The present invention is based on the discovery of a human Emr1-like G protein-coupled receptor. The human Emr1-like G protein-coupled receptor, an encoding DNA, and an antibody which specifically binds the protein are useful in the diagnosis, prognosis, treatment and evaluation of therapies for respiratory, inflammatory, and immunological disorders.

The invention provides a cDNA which encodes a protein having an amino acid sequence of SEQ ID NO:1. The invention also provides cDNAs having the nucleic acid sequences of SEQ ID NOs:2-10. The invention additionally provides compositions comprising the cDNAs or complements thereof and a labeling moiety which may be used in methods of the invention, on a substrate, or as probes. The invention further provides a vector containing the cDNA, a host cell containing the vector, and a method for using the host cell to make the human protein. The invention still further provides a transgenic cell line or organism comprising the vector containing a cDNA selected from SEQ ID NO:2-10. In a second aspect, the invention provides a cDNA or the complement thereof which can be used in methods of detection, screening, and purification. In a further aspect, the cDNA is a single-stranded RNA or DNA molecule, a peptide nucleic acid, a branched nucleic acid, and the like.

The invention provides a method for using a cDNA to detect differential expression of a nucleic acid in a sample comprising hybridizing a cDNA to the nucleic acids, thereby forming hybridization complexes and comparing hybridization complex formation with at least one standard, wherein the comparison indicates differential expression of the cDNA in the sample. In one aspect, the method of detection further comprises amplifying the nucleic acids of the sample prior to hybridization. In another aspect, the method showing differential expression of the cDNA is used to diagnose a respiratory, inflammatory, or immunological disorder.

The invention additionally provides a method for using a cDNA of the invention to screen a plurality of molecules or compounds to identify or purify at least one ligand which specifically binds the cDNA, the method comprising combining the cDNA with the molecules or compounds under conditions allowing specific binding, and detecting specific binding to the cDNA, thereby identifying or purifying a ligand which binds the cDNA. In one aspect, the molecules or compounds are selected from aptamers, DNA molecules, RNA molecules, peptide nucleic acids, artificial chromosome constructions, peptides, transcription factors, repressors, and regulatory molecules.

The invention provides a purified protein or a portion thereof selected from the group consisting of a protein having an amino acid sequence of SEQ ID NO:1, a protein with 90% identity to the amino acid sequence of SEQ ID NO:1 which binds R5833 antiserum, an antigenic determinant of SEQ ID NO:1, and a biologically active portion of SEQ ID NO:1. The invention also provides a composition comprising the purified protein and a labeling moiety or a pharmaceutical carrier. The invention still further provides a method for using a protein to screen a library or a plurality of molecules or compounds to identify or purify at least one ligand, the method comprising combining the protein with the molecules or compounds under conditions to allow specific binding and detecting specific binding, thereby identifying or purifying a ligand which specifically binds the protein. In one aspect, the molecules or compounds are selected from DNA molecules, RNA molecules, peptide nucleic acids, peptides, proteins, mimetics, agonists, antagonists, inhibitors, and drugs. In another aspect, the ligand is used to treat a subject with a respiratory, inflammatory, and immunological disorder. The invention yet still further provides an array containing the human Emr1-like G protein-coupled receptor.

The invention provides a method for testing a molecule or compound for effectiveness as an agonist comprising exposing a sample comprising a protein to a molecule or compound, and detecting agonist activity in the sample. The invention also provides method for testing a molecule or compound for effectiveness as an antagonist comprising exposing a sample comprising a protein to a molecule or compound and detecting antagonist activity in the sample.

The invention provides a method for using a protein to screen a plurality of antibodies to identify an antibody which specifically binds the protein comprising contacting a plurality of antibodies with the protein under conditions to form an antibody:protein complex, and dissociating the antibody from the antibody:protein complex, thereby obtaining antibody which specifically binds the protein.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention further provides purified antibodies which bind specifically to a protein. The invention also provides a method for using an antibody to detect expression of a protein in a sample, the method comprising combining the antibody with a sample under conditions for formation of antibody:protein complexes; and detecting complex formation, wherein complex formation indicates expression of the protein in the sample. In one aspect, the amount of complex formation when compared to standards is diagnostic of a respiratory, inflammatory or immunological disorder.

The invention still further provides a method for immunopurification of a protein comprising attaching an antibody to a substrate, exposing the antibody to a sample containing protein under conditions to allow antibody:protein complexes to form, dissociating the protein from the complex, and collecting purified protein. The invention yet still further provides an array containing an antibody which specifically binds the human Emr1-like G protein-coupled receptor.

The invention provides a method for inserting a heterologous marker gene into the genomic DNA of a mammal to disrupt the expression of the endogenous polynucleotide. The invention also provides a method for using a cDNA to produce a model system, the method comprising constructing a vector containing a cDNA selected from SEQ ID NOs:2-10, transforming the vector into an embryonic stem cell, selecting a transformed embryonic stem cell, microinjecting the transformed embryonic stem cell into a blastocyst, thereby forming a chimeric blastocyst, transferring the chimeric blastocyst into a pseudopregnant dam, wherein the dam gives birth to a chimeric offspring containing the cDNA in its germ line, and breeding the chimeric mammal to produce a homozygous, model system.

### BRIEF DESCRIPTION OF THE FIGURES AND TABLES

FIGS. 1A-1H show the amino acid sequence of SEQ ID NO:1 and the nucleic acid sequence encoding EGPCR (SEQ ID NO:2). The alignment was produced using MACDNASIS PRO software (Hitachi Software Engineering, San Bruno Calif.).

FIGS. 2A-2E show the amino acid sequence alignments among EGPCR (429905; SEQ ID NO:1), human Emr1 (g784994; SEQ ID NO:11), and human orphan Emr1-like GPCR (g2935597; SEQ ID NO:12), produced using the multisequence alignment program of LASERGENE software (DNASTAR, Madison Wis.).

FIG. 3 shows the northern blot autoradiograph of EGPCR expression in different human tissue samples. The tissue source of the mRNA is listed above each lane. The position of standard molecular weight polynucleotide markers are to the left of the autoradiograph. The position of the two EGPCR mRNA species are shown by two arrows to the right of the autoradiograph.

FIG. 4 shows western blot immunoautoradiographs of EGPCR in HEK 293 cell lysates following transfection of HEK 293 cells with a vector containing an EGPCR fragment-green fluorescent protein construct (EF-GFP) or Myc-EF-GFP. The blots were immunostained using either anti-EGPCR antibody (Anti429905) or anti-Myc antibody (Anti-Myc). The description above each lane describes which plasmid construct was used to transfect the cells. The position of standard molecular weight protein markers are listed to the left of the autoradiogaphs.

FIG. 5 shows western blot immunoautoradiographs of EF-GFP-transfected HEK 293 cell lysate treated with or without N-glycosylase. The blots were immunostained using either anti-EGPCR antibody (α-429905; R5833) or anti-Myc antibody (α-Myc). The description above each lane describes which plasmid construct was used to transfect the cells. Samples were untreated (−) or treated (+) with N-glycosylase. The position of standard molecular weight protein markers are the left of the autoradiograph.

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody of diagnostic or therapeutic interest. The arrangement of two to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

The “complement” of a cDNA of the Sequence Listing refers to a nucleic acid molecule which is completely complementary to the cDNA over its full length and which will hybridize to the cDNA or an mRNA under conditions of maximal stringency.

“cDNA” refers to an isolated polynucleotide, nucleic acid molecule, or any fragment or complement thereof. It may have originated recombinantly or synthetically, may be double-stranded or single-stranded, represents coding and noncoding 3′ or 5′ sequence, and generally lacks introns.

A “composition” refers to the polynucleotide and a labeling moiety; a purified protein and a pharmaceutical carrier or a heterologous, labeling or purification moiety; an antibody and a labeling moiety; and the like.

“Derivative” refers to a cDNA or a protein that has been subjected to a chemical modification. Derivatization of a cDNA can involve substitution of a nontraditional base such as queosine or of an analog such as hypoxanthine. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or up-regulated or a decreased or down-regulated expression as detected by presence, absence, or at least two-fold change in the amount or abundance of a transcribed messenger RNA or translated protein in a sample.

“Disorders” refer to conditions, diseases of disorders of the respiratory or immune systems, and particularly to “eosinophilia”; the dramatic increase in the number of eosinophils per microliter of blood observed in hypereosinophilic syndrome (hypereosinophilia), allergies, COPD, and asthma; and in lung cancer.

“EGPCR” refers to purified protein obtained from any species, particularly a mammalian species, including bovine, ovine, porcine, murine, equine, and preferably the human species, from any source, whether natural, synthetic, semi-synthetic, or recombinant.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. Hybridization conditions, degree of complementarity and the use of nucleotide analogs affect the efficiency and stringency of hybridization reactions.

“Labeling moiety” refers to any reporter molecule or visible or radioactive label than can be attached to or incorporated into a cDNA, protein, or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which may be used in hybridization or amplification technologies or in regulation of replication, transcription or translation. Equivalent terms are amplimer, primer, and oligomer.

An “oligopeptide” is an amino acid sequence from about five residues to about 15 residues that is used as part of a fusion protein to produce an antibody.

“Portion” refers to any part of a protein used for any purpose; but particularly, to an epitope for the screening of ligands or for the production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR).

“Purified” refers to any molecule or compound that is separated from its natural environment and is from about 60% free to about 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, mRNA, or cDNA in solution or bound to a substrate; a cell; a tissue; a tissue print; a fingerprint, buccal cells, semen, skin, or hair; and the like.

“Similarity” refers to the quantification (usually percentage) of nucleotide or residue matches between at least two sequences aligned using a standard algorithm such as Smith-Waterman alignment (Smith and Waterman (1981) J Mol Biol 147:195-197) or BLAST2 (Altschul et al. (1997) Nucleic Acids Res 25:3389-3402). BLAST2 may be used in a reproducible way to insert gaps in one of the sequences in order to optimize alignment and to achieve a more meaningful comparison between them. Particularly in proteins, similarity is greater than identity in that conservative substitutions (for example, valine for leucine or isoleucine) are counted in calculating the reported percentage. Substitutions which are considered to be conservative are well known in the art.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs, proteins, or antibodies are bound and includes magnetic or nonmagnetic beads; capillaries; chips; fibers; filters; gels; membranes; microparticles with a variety of surface forms including wells, trenches, pins, channels and pores; phages; plates; polymers; slides; glass, metal, paper, plastic rubber or other tubing; and wafers.

A “transcript image” is a profile of gene transcription activity based on the reverse transcription of mRNAs and the production and sequencing of cDNAs from a particular tissue at a particular time.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The invention is based on the discovery of a new human Emr1-like G protein-coupled receptor (EGPCR), the cDNAs encoding EGPCR, and the use of these compositions for the diagnosis, treatment, or prevention of respiratory, inflammatory, and immunological disorders, particularly eosinophilia and lung cancer. U.S. Ser. No. 09/368,394, filed Aug. 4, 1999, is incorporated by reference herein.

The cDNA encoding EGPCR of the present invention was first identified in Incyte clone 429905H1 from the EOSINOT03 eosinophil cDNA library. The 3350 base pair, full length, nucleic acid sequence of SEQ ID NO:2 was derived as discussed in EXAMPLE III, but it can also be assembled using overlapping sequences, SEQ ID NOs:3-10, from Incyte clones 429905H1 (EOSINOT03), 1837630F6 (EOSITXT01), 8009216H1(NOSEDIC02), 1837630T6 (EOSITXT01), 2438817F7 (EOSITXT01), 320551R1(EOSIHET02), 524802R6 (CARCTXT01), and 7749732H1(NOSEDIN01), respectively. It should be noted that the majority of these cDNAs came from eosinophil libraries.

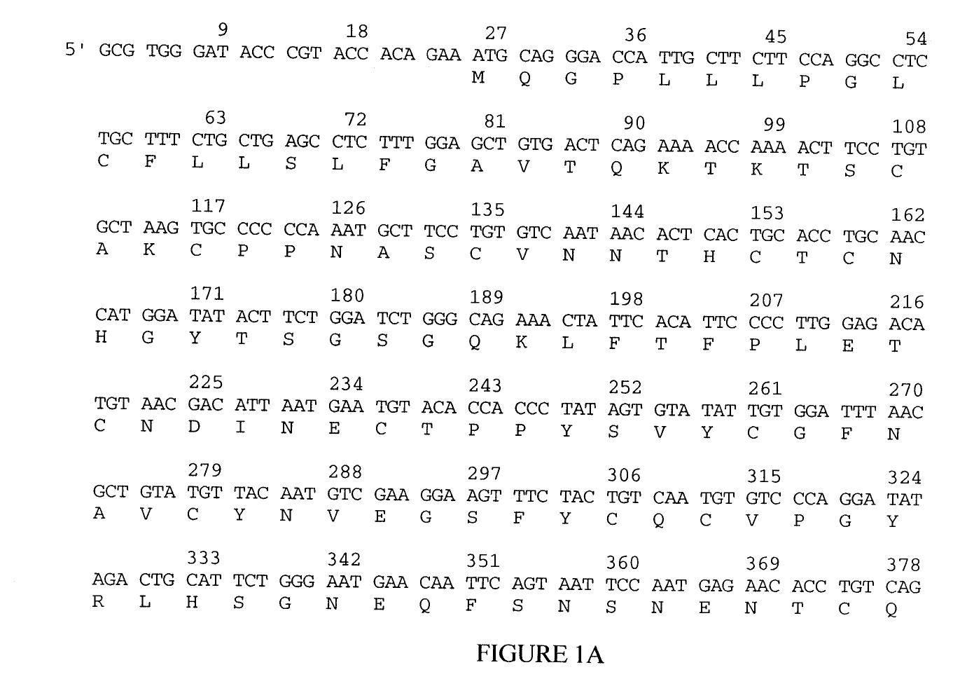

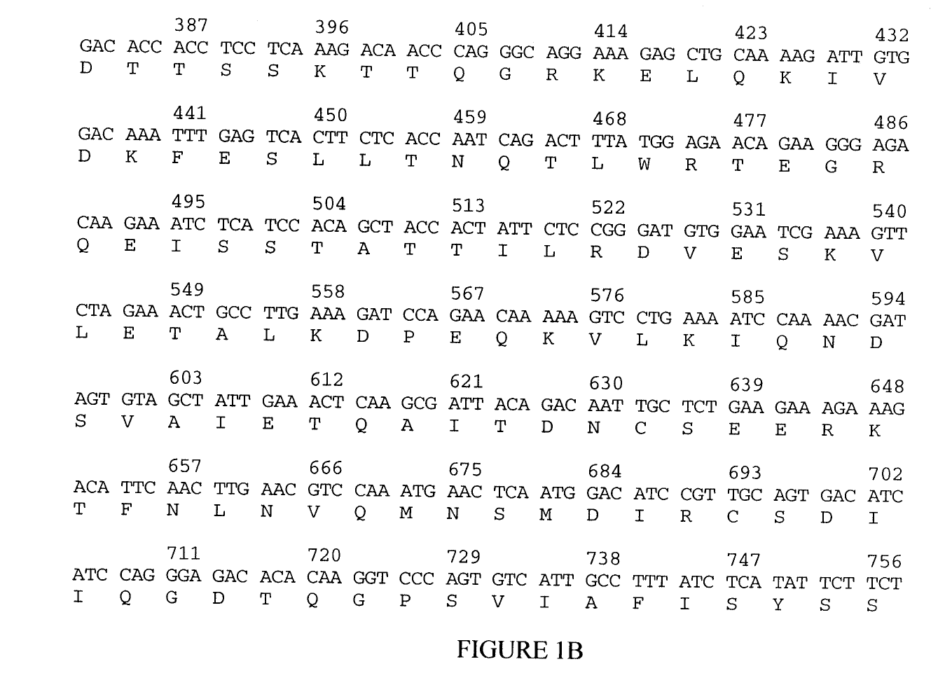

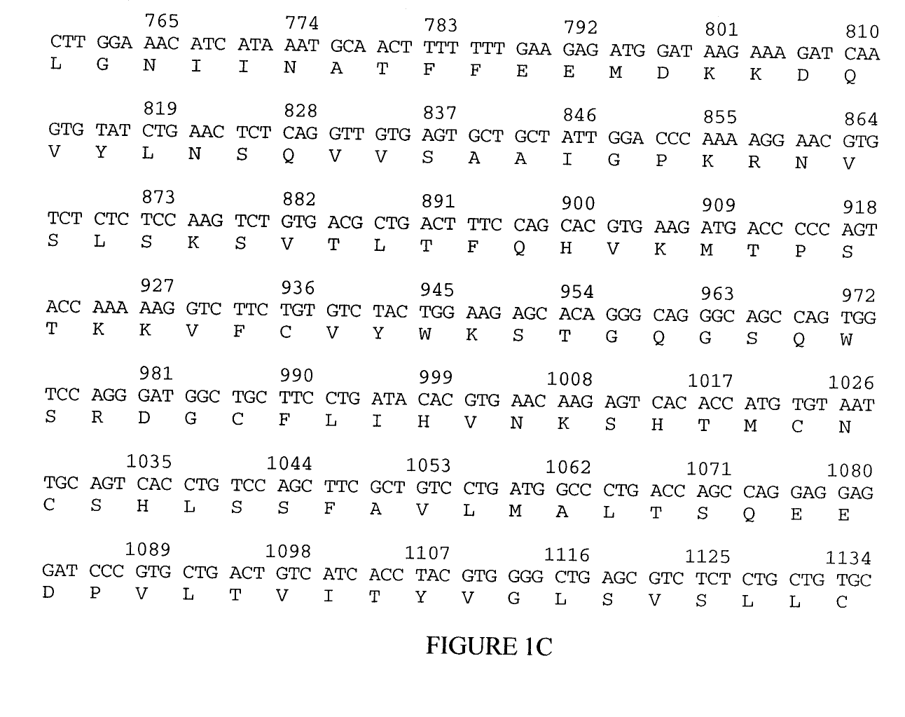

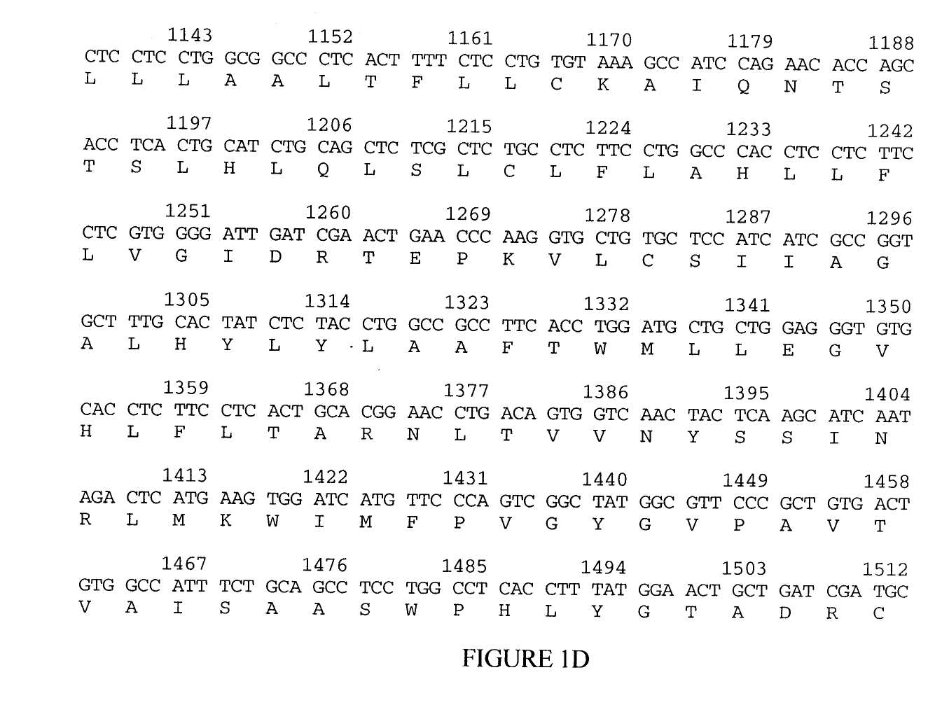

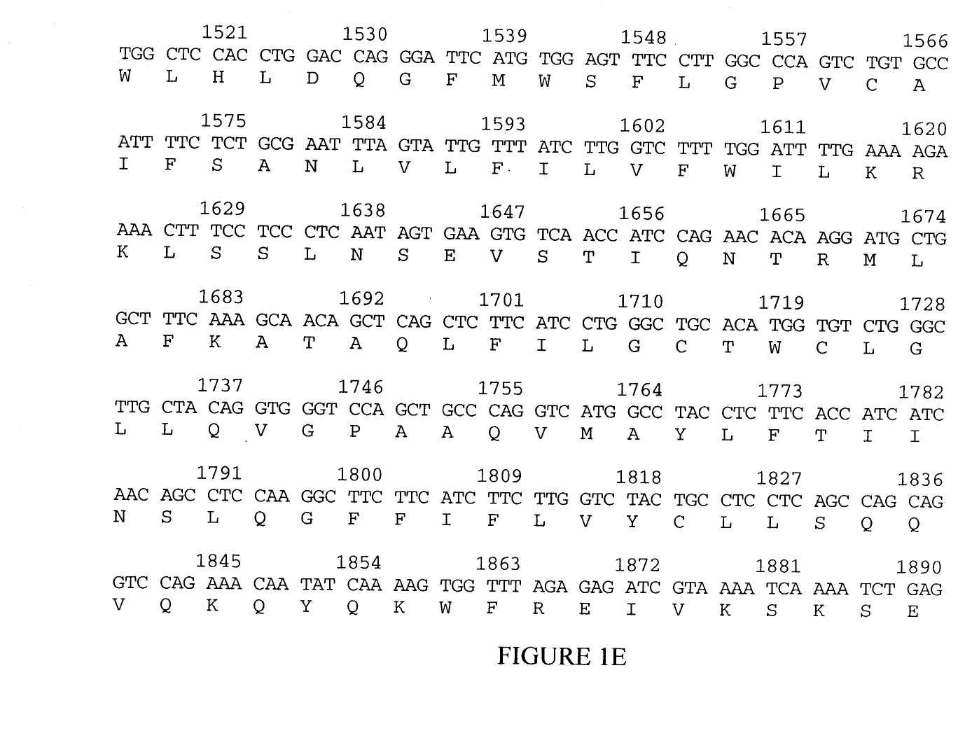

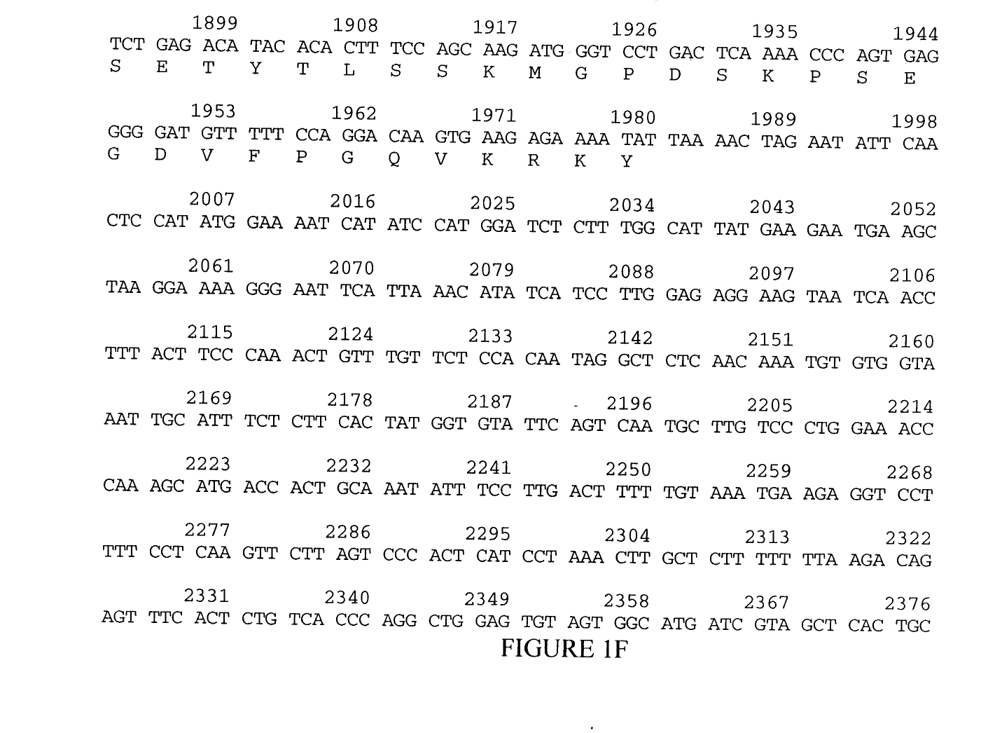

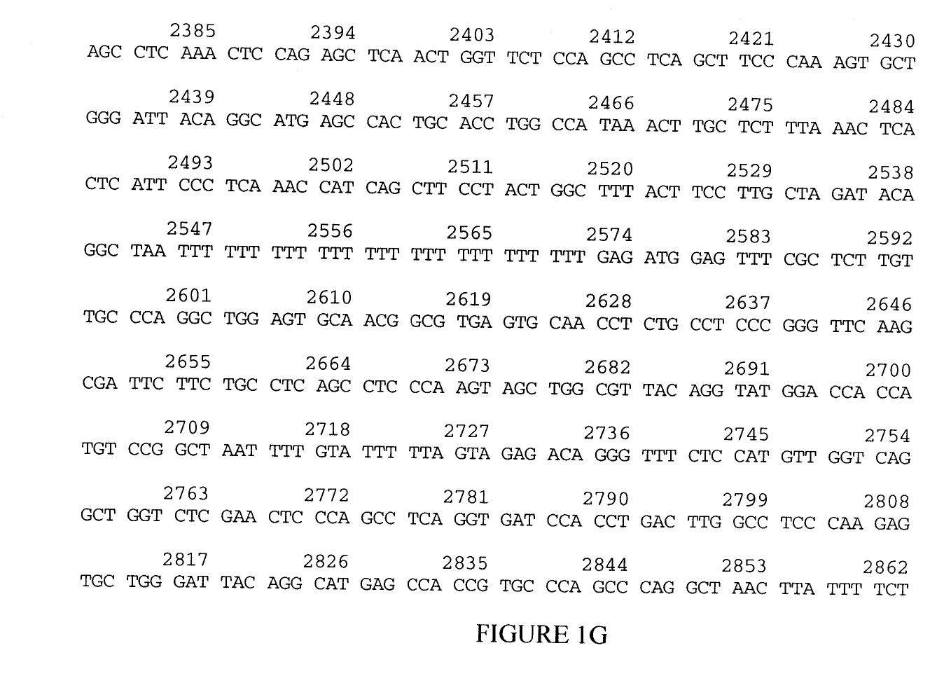

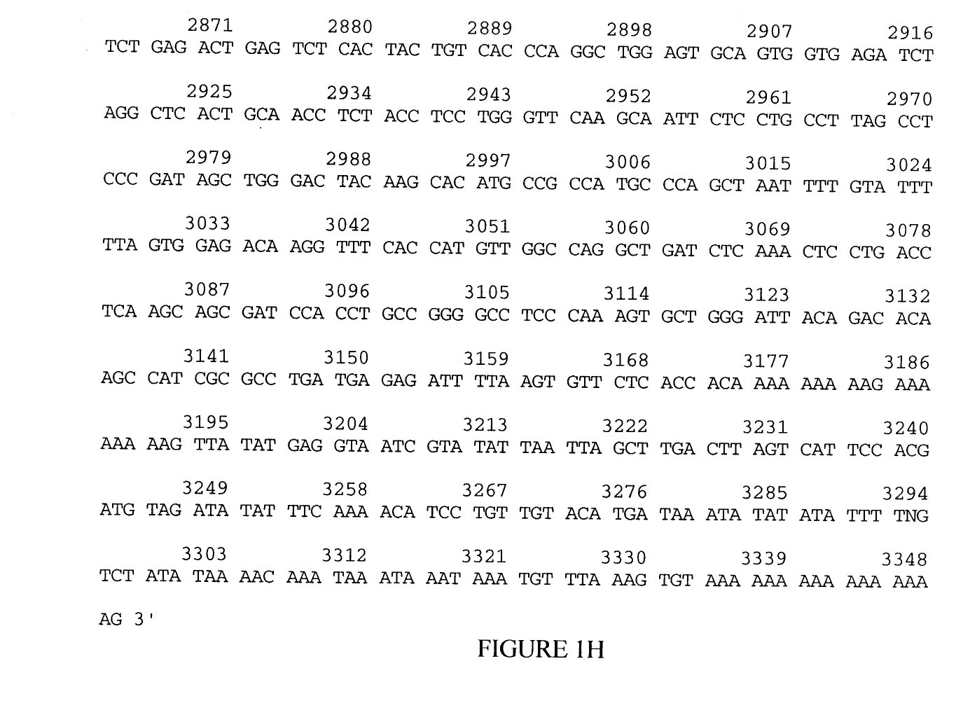

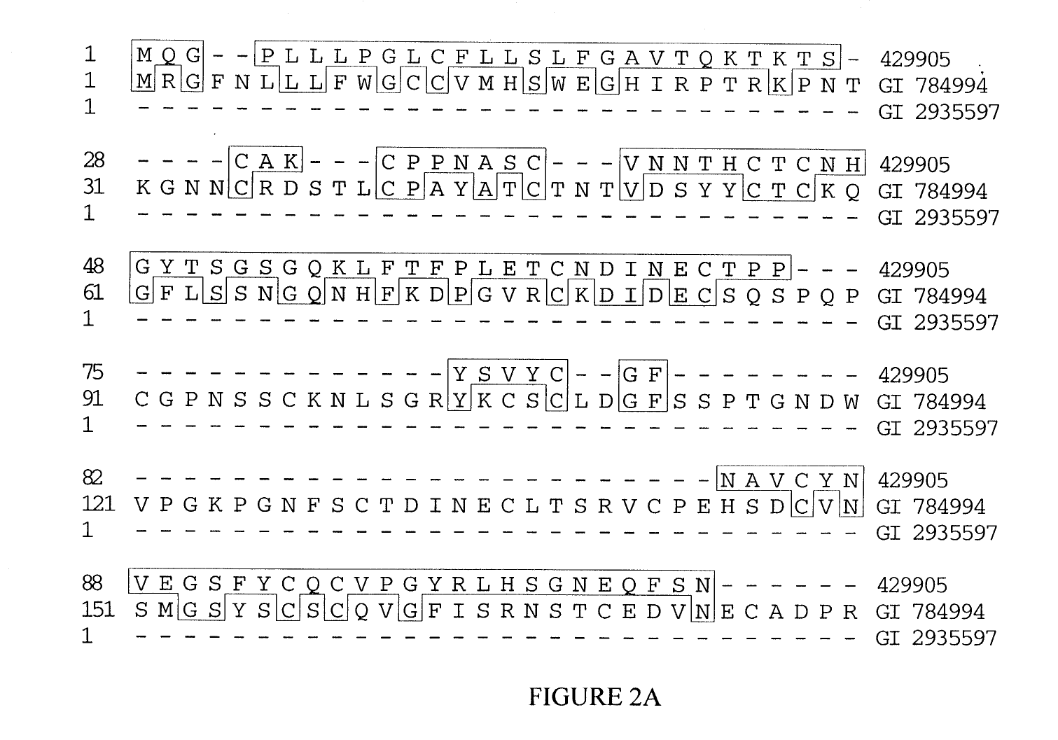

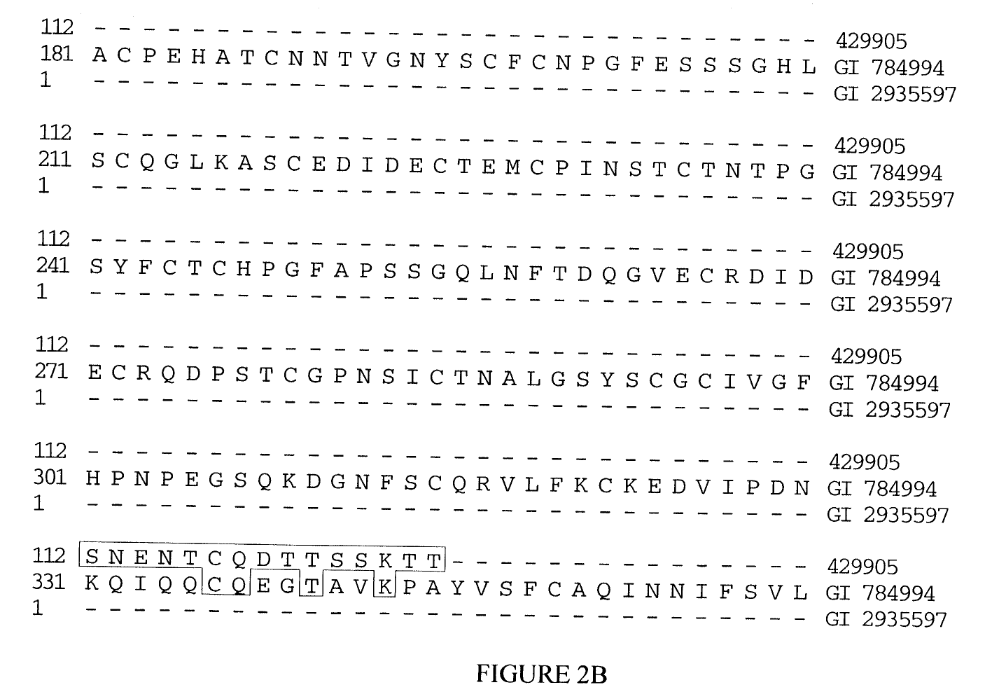

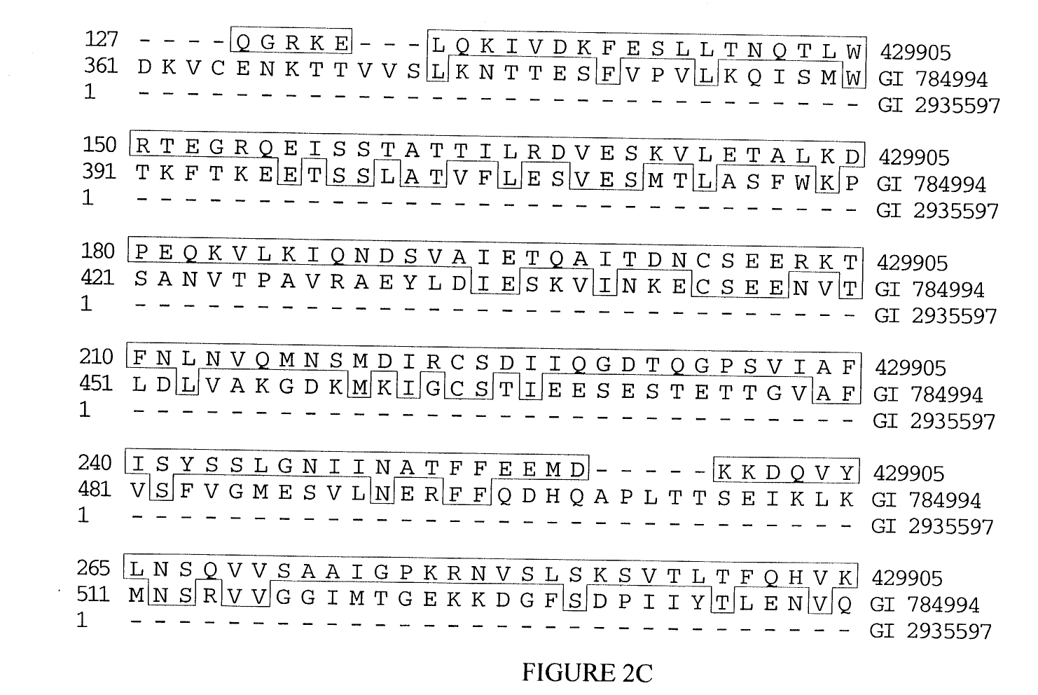

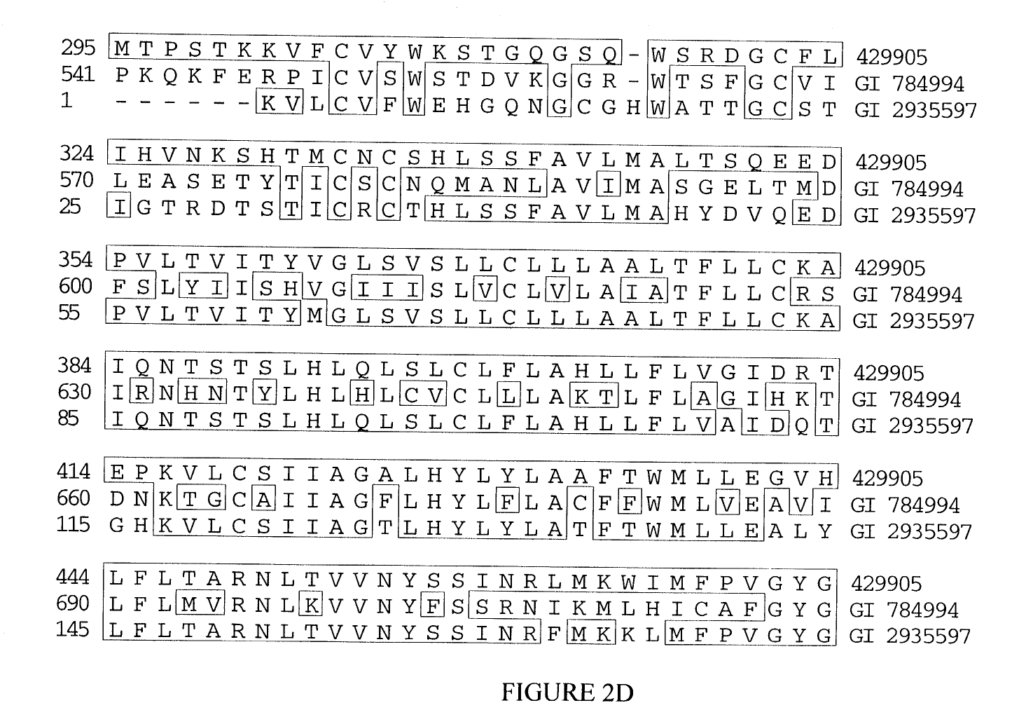

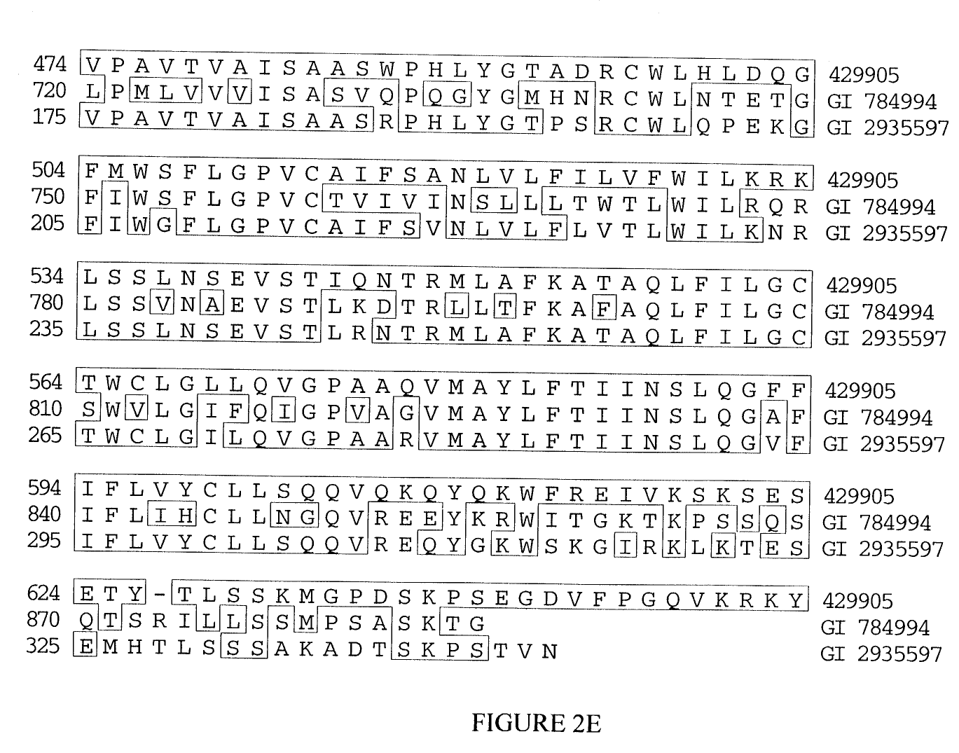

In one embodiment, the invention encompasses a protein comprising the amino acid sequence of SEQ ID NO:1, as shown in FIGS. 1A-1H. EGPCR is 652 amino acids in length and has a predicted molecular mass of approximately 72.6 kDa. EGPCR has twelve potential N-glycosylation sites at residues N34, N39, N145, N189, N202, N250, N279, N327, N334, N386, N450, and N455; one potential cAMP- and cGMP-dependant protein kinase phosphorylation site at residue S535; nine potential casein kinase II phosphorylation sites at residues T64, S104, T116, T252, T348, S349, S619, S621, and S639; one potential glycosaminoglycan attachment site at residue S51; six potential protein kinase C phosphorylation sites at residues T21, S122, S298, T299, T447, and S629; one potential tyrosine kinase phosphorylation site at residue Y626; one potential aspartic acid and asparagine hydroxylation site at residue N87; two Type II EGF-like signatures from residues C28 to C66 and C72 to C117; one Typ I EGF-like signature from residues C304 to C370; and the G-protein coupled receptor motifs from residues C381 to I410, C419 to A448, M505 to S535, and S588 to F613. As shown in FIGS. 2A-2E, EGPCR has chemical and structural similarity with human Emr1 (g784994; SEQ ID NO:11), and human orphan Emr1-like GPCR (g2935597; SEQ ID NO:12). In particular, EGPCR and human orphan Emr1-like GPCR share 57% identity, two potential N-glycosylation sites, the potential cAMP- and cGMP-dependant protein kinase phosphorylation site, two potential casein kinase II phosphorylation sites, three potential protein kinase C phosphorylation sites, the aspartic acid and asparagine hydroxylation site, the type I EGF-like signature, and the G-protein coupled receptor motifs. A fragment of SEQ ID NO:2 from about nucleotide 1399 to about nucleotide 1428 is useful, for example, for designing oligonucleotides or as a hybridization probe.

A hydropathy plot analysis of EGPCR revealed an N-terminal signal peptide followed by an extracellular domain of 350 residues, seven hydrophobic potential transmembrane regions, and a C-terminal 320 amino acid residues. The structural features of a GPCR and sequence homology placed EGPCR in the recently defined novel EGF-TM7 receptor subfamily. This subfamily is related to the hormone receptor gene family and includes receptors for glucagon, calcitonin, corticotropin releasing hormone, secretin, VIP, and diuretic hormone. The expression pattern of EGPCR was examined by electronic northern analysis of Incyte cDNA libraries isolated from various tissues and cell types. The first column of the table below lists tissue expression of EGPCR and fraction of total tissue which expresses SEQ ID NO:1. The second column lists the disease class and fraction of total disease tissues that express SEQ ID NO:1.

| Tissue Expression | Disease Class |
|---|---|
| Hematopoietic/Immune (0.409) | Inflammation/Trauma (0.477) |
| Cardiovascular (0.167) | Cancer (0.227) |
| Reproductive (0.129) | |

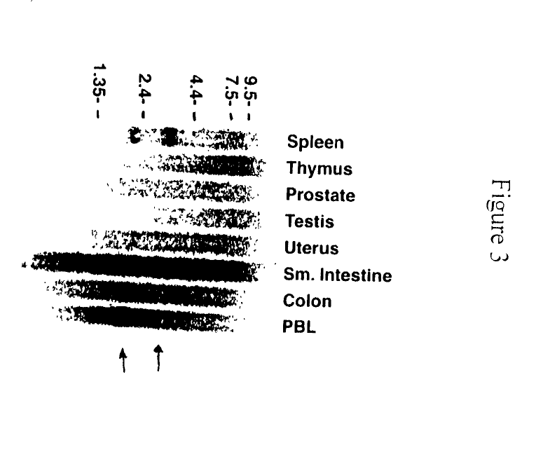

In EGPCR was highly expressed in eosinophils when compared to its low expression in activated monocytes and neutrophils. In addition, the expression pattern of EGPCR was examined using membrane-based northern analysis of RNA isolated from various tissues and cell types. FIG. 3 shows that EGPCR mRNA was expressed at low level in spleen, peripheral blood leukocytes, and bone marrow. This pattern was consistent with expression in cells or tissues involved in immune response. Two mRNA species of 3.5 kb and 1.8 kb were observed in tissues expressing EGPCR. In peripheral blood leukocytes, the 1.8 kb species seems to be expressed at a slightly higher levels than in other tissues. It is conceivable that these two species are the results of alternative splicing and the 1.8 kb mRNA species may encode a splice variant of the gene.

A transcript image of gene expression is shown in EXAMPLE VIII. When used in a tissue-specific manner with peripheral blood or lung tissue, SEQ ID NOs:1 and 2 are diagnostic of specific respiratory and immunological disorders, eosinophilia and lung cancer, respectively.

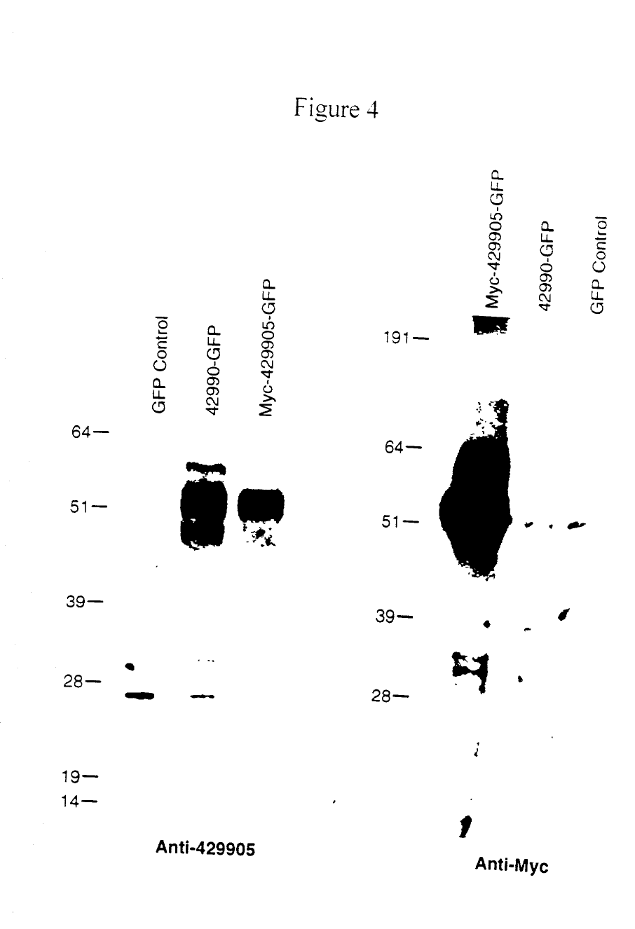

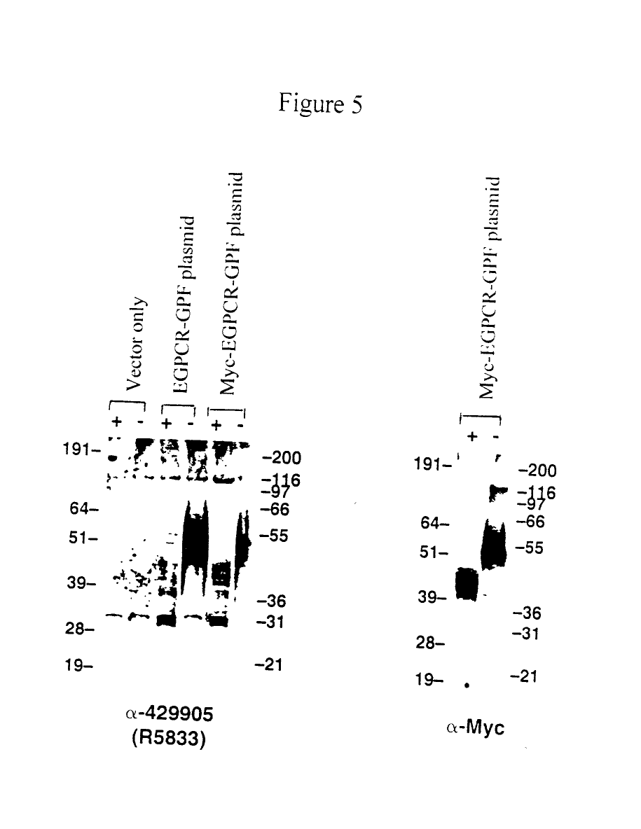

An EGPCR fragment-green fluorescent protein (GFP) conjugate was expressed in HEK 293 cells following transfection with EGPCR fragment-GFP-containing vector. As shown in FIG. 4, cell lysates from transfected HEK 293 cells contained a 51-53 kDa band of protein which bound to anti-EGPCR antibody. As shown in FIG. 5, cell lysates from transfected HEK 293 cells that had been treated with N-glycosidase contained an anti-EGPCR-binding protein of approximately 31 kDa. The difference in size between treated and native EGPCR was attributed to postranslational glycosylation.

It will be appreciated by those skilled in the art that as a result of the degeneracy of the genetic code, a multitude of cDNAs encoding human EGPCR, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring EGPCR, and all such variations are to be considered as being specifically disclosed.

The cDNAs of SEQ ID NOs:2-10 may be used in hybridization, amplification, and screening technologies to identify and distinguish among SEQ ID NOs:2-10 and related molecules in a sample. The cDNAs may also be used to produce transgenic cell lines or organisms which are model systems for respiratory, inflammatory, or immunological disorders and upon which the toxicity and efficacy of potential therapeutic treatments may be tested. Toxicology studies, clinical trials, and subject/patient treatment profiles may be monitored using the cDNAs, proteins, antibodies and molecules and compounds identified using the cDNAs and proteins of the present invention.

Characterization and Use of the Invention

cDNA libraries

In a particular embodiment disclosed herein, mRNA is isolated from cells and tissues using methods which are well known to those skilled in the art and used to prepare the cDNA libraries. The Incyte cDNAs were isolated from cDNA libraries prepared as described in the EXAMPLES. The consensus sequences are chemically and/or electronically assembled from fragments including Incyte cDNAs and extension and/or shotgun sequences using computer programs such as PHRAP (P Green, University of Washington, Seattle Wash.), and the AUTOASSEMBLER application (Applied Biosystems (ABI), Foster City Calif.). After verification of the 5′ and 3′ sequence, at least one of the representative cDNAs which encode the EGPCR is designated a reagent. In this case, the reagent cDNA is SEQ ID NO:4, Incyte clone 1837630F6 from the eosinophil cDNA library, EOSITXT01. These reagent cDNAs are also used in the construction of human microarrays and are represented among the sequences on the Human Genome Gem Arrays (Incyte Genomics).

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Phanmacia Biotech (APB), Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 system (Hamilton, Reno Nev.) and the DNA ENGINE thermal cycler (MJ Research, Watertown Mass.). Machines commonly used for sequencing include the PRISM 3700, 377 or 373 DNA sequencing systems (ABI), the MEGABACE 1000 DNA sequencing system (APB), and the like. The sequences may be analyzed using a variety of algorithms well known in the art and described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, or deleted sequences can be removed or restored, respectively, organizing the incomplete assembled sequences into finished sequences.

Extension of a Nucleic Acid Sequence

The sequences of the invention may be extended using various PCR-based methods known in the art. For example, the XL-PCR kit (ABI), nested primers, and commercially available cDNA or genomic DNA libraries may be used to extend the nucleic acid sequence. For all PCR-based methods, primers may be designed using commercially available software to be about 22 to 30 nucleotides in length, to have a GC content of about 50% or more, and to anneal to a target molecule at temperatures from about 55 C. to about 68 C. When extending a sequence to recover regulatory elements, it is preferable to use genomic, rather than cDNA libraries.

Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the EGPCR, allelic variants, or related molecules. The probe may be DNA or RNA, may be single-stranded, and should have at least 50% sequence identity to a nucleic acid sequence selected from SEQ ID NOs:2-10. Hybridization probes may be produced using oligolabeling, nick translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using commercially available kits.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60 C., which permits the formation of a hybridization complex between nucleic acid sequences that contain some mismatches. Subsequent washes are performed at higher stringency with buffers such as 0.2×SSC with 0.1% SDS at either 45 C. (medium stringency) or 68 C. (high stringency). At high stringency, hybridization complexes will remain stable only where the nucleic acids are completely complementary. In some membrane-based hybridizations, preferably 35% or most preferably 50%, formamide can be added to the hybridization solution to reduce the temperature at which hybridization is performed, and background signals can be reduced by the use of detergents such as Sarkosyl or TRITON X-100 (Sigma-Aldrich, St Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays incorporating cDNAs, proteins, or antibodies may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Proteins may be used to identify ligands, to investigate protein:protein interactions, or to produce a proteomic profile of gene expression (i.e., to detect and quantify expression of a protein in a sample). Antibodies may be also be used produce a proteomic profile of gene expression. Such arrays may be used to determine gene function; to understand the genetic basis of a condition, disease, or disorder; to diagnose a condition, disease, or disorder; and to develop and monitor the activities of therapeutic agents. (See, e.g., Brennan et al. (1995) U.S. Pat. No. 5,474,796; Schena et al. (1996) Proc Natl Acad Sci 93:10614-10619; Heller et al. (1997) Proc Natl Acad Sci 94:2150-2155; Heller et al. (1997) U.S. Pat. No. 5,605,662; and deWildt et al. (2000) Nature Biotechnol 18:989-994.)

Hybridization probes are also useful in mapping the naturally occurring genomic sequence. The probes may be hybridized to a particular chromosome, a specific region of a chromosome, or an artificial chromosome construction. Such constructions include human artificial chromosomes (HAC), yeast artificial chromosomes (YAC), bacterial artificial chromosomes (BAC), bacterial P1 constructions, or the cDNAs of libraries made from single chromosomes.

Expression

Any one of a multitude of cDNAs encoding the EGPCR may be cloned into a vector and used to express the protein, or portions thereof, in host cells. The nucleic acid sequence can be engineered by such methods as DNA shuffling, as described in U.S. Pat. No. 5,830,721, and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mammalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional PBLUESCRIPT vector (Stratagene, La Jolla Calif.) or PSPORT1 plasmid (Life Technologies). Introduction of a nucleic acid sequence into the multiple cloning site of these vectors disrupts the lacZ gene and allows calorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6xHis, FLAG, MYC, and the like. GST and 6xHis are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amnino acids, and washing with dichloromethane and/or N, N-dimethylformamide. The peptide is cleaved between the peptide carboxy terminus and the linker group to yield a peptide acid or amide. These processes are described in the Novabiochem 1997/98 Catalog and Peptide Synthesis Handbook (San Diego Calif. pp. S1-S20). Automated synthesis may also be carried out on machines such as the ABI 43 1A peptide synthesizer (ABI). A protein or portion thereof may be purified by preparative high performance liquid chromatography and its composition confirmed by amino acid analysis or by sequencing (Creighton (1984) Proteins, Structures and Molecular Properties, W H Freeman, New York N.Y.).

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two main functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich, St. Louis Mo.), and dinitrophenol may be used to increase immunological response. In humans, BCG (bacilli Calmette-Guerin) and Corynebacterium parvum are preferable. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al (1975) Nature 256:495-497; Kozbor et al (1985) J Immunol Methods 81:31-42; Cote et al (1983) Proc Natl Acad Sci 80:2026-2030; and Cole et al (1984) Mol Cell Biol 62:109-120).

“Chimeric antibodies” may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are preferred for use in immunoassays in which the protein-antibody complex must withstand rigorous manipulations. Low-affinity antibody preparations with Kₐ ranging from about 10⁶ to 10 ⁷ L/mole are preferred for use in immunopurification and similar procedures which ultimately require dissociation of the protein, preferably in active form, from the antibody (Catty (1988) Antibodies, Volume I: A Practical Approach, IRL Press, Washington DC; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are widely available (Catty, supra; Ausubel (supra) pp. 11.1-11.31)

Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison Wis.) for incorporation of a labeled nucleotide such as ³²P-dCTP (APB), Cy3-dCTP or Cy5-dCTP (Operon Technologies, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

Diagnostics

Nucleic Acid Assays

The cDNAs, fragments, oligonucleotides, complementary RNA and DNA molecules, and PNAs may be used to detect and quantify differential gene expression for diagnostic purposes. Respiratory, inflammatory, or immunological disorders associated with differential expression of SEQ ID NO:2-10 specifically include eosinophilia and lung cancer. The diagnostic assay may use hybridization or quantitative PCR to compare gene expression in a biological sample from a patient to standard samples in order to detect differential gene expression. Qualitative and quantitative methods for this comparison are commercially available and well known in the art.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

In order to provide standards for establishing differential expression, normal and diseased tissue expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Proteomic/Immunological Assays

Detection and quantification of a protein using either labeled amino acids or antibodies which specifically bind the protein are known in the art. Examples of such techniques include two-dimensional polyacrylamide gel electrophoresis, enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. A two-site, monoclonal-based immunoassay utilizing antibodies reactive to two non-interfering epitopes is preferred, but a competitive binding assay may be employed (Coligan et al. (1997) Current Protocols in Immunology, Wiley-Interscience, New York N.Y.; Pound (1998) Immunochemical Protocols, Humana Press, Totowa N.J.). These assays and their quantitation against purified, labeled standards are well known in the art (Ausubel, supra, units 10.1-10.6).

Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Proteomic and immunological methods are also useful for showing differential gene expression associated with the diagnosis of respiratory, inflammatory, and immunological disorders, particularly eosinophilia and lung cancer.

Therapeutics

As described in THE INVENTION section, chemical and structural similarity, in particular the sequences and specific motifs, exist between regions of the EGPCR (SEQ ID NO:1), human Emr1 (g784994; SEQ ID NO:11), and human orphan Emr1-like GPCR (g2935597; SEQ ID NO:12). In addition, differential expression was demonstrated in respiratory, inflammatory, and immunological disorders using northern analyses and transcript imaging. Exemplary and confirmatory transcript images for differential expression of EGPCR are shown in EXAMPLE VIII. Thus, EGPCR and it encoding cDNA clearly play a role in respiratory, inflammatory, and immunological disorders particularly eosinophilia and lung cancer.

In one embodiment, increased expression of the protein may be treated by the delivery of an inhibitor, antagonist, antibody and the like or a pharmaceutical composition containing one or more of these molecules. Such delivery may be effected by methods well known in the art and may include delivery by an antibody specifically targeted to the diseased tissue. Neutralizing antibodies which inhibit dimer formation are generally preferred for therapeutic use.

In another embodiment, decreased expression of the protein in a disorder may be treated by the delivery of the protein, an agonist, enhancer and the like or a pharmaceutical composition containing one or more of these molecules. Such delivery may be effected by methods well known in the art and may include delivery of a therapeutic agent by an antibody specifically targeted to the diseased tissue.

Any of the compositions containing the cDNA, protein, or antibody may be administered in combination with other therapeutic agents. Selection of the agents for use in combination therapy may be made by one of ordinary skill in the art according to conventional pharmaceutical principles. A combination of therapeutic agents may act synergistically to affect treatment of a particular cancer at a lower dosage of each agent alone.

Modification of Gene Expression Using Nucleic Acids

Gene expression may be modified by designing complementary or antisense molecules (DNA, RNA, or PNA) to the control, 5′, 3′, or other regulatory regions of the gene encoding EGPCR. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio- groups renders the molecule less available to endogenous endonucleases.

Screening and Purification Assays

The cDNA encoding EGPCR may be used to screen a plurality, or a library, of molecules or compounds for specific binding affinity. The libraries may be aptamers, DNA molecules, RNA molecules, PNAs, peptides, proteins such as transcription factors, enhancers, or repressors, and other ligands which regulate the activity, replication, transcription, or translation of the endogenous gene. The assay involves combining a cDNA with a plurality of molecules or compounds under conditions allowing specific binding, and detecting specific binding to identify at least one molecule which specifically binds the single-stranded or double-stranded molecule.

In one embodiment, the cDNA of the invention may be incubated with a plurality of purified molecules or compounds and binding activity determined by methods well known in the art, e.g., a gel-retardation assay (U.S. Pat. No. 6,010,849) or a commercially available reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using a chaotropic agent to separate the protein from the purified ligand.

In a preferred embodiment, EGPCR may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify DNA molecules, RNA molecules, peptide nucleic acids, peptides, proteins, mimetics, agonists, antagonists, antibodies, immunoglobulin, inhibitors, and drugs or any other ligand, which specifically binds the protein.

In one aspect, this invention comtemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a model system to evaluate their toxicity, diagnostic, or therapeutic potential.

Pharmacology

Pharmaceutical compositions contain active ingredients in an effective amount to achieve a desired and intended purpose and a pharmaceutical carrier. The determination of an effective dose is well within the capability of those skilled in the art. For any compound, the therapeutically effective dose may be estimated initially either in cell culture assays or in animal models. The animal model is also used to achieve a desirable concentration range and route of administration. Such information may then be used to determine useful doses and routes for administration in humans.

A therapeutically effective dose refers to that amount of protein or inhibitor which ameliorates the symptoms or condition. Therapeutic efficacy and toxicity of such agents may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions which exhibit large therapeutic indexes are preferred. The data obtained from cell culture assays and animal studies are used in formulating a range of dosage for human use.

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

Transeenic Animal Models

Transgenic rodents that over-express or under-express a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gen, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NBPs, early research and toxicological studies are usually carried out in rodent models. In studies using behavioral measures such as drug addiction, NHs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

I cDNA Library Construction

The eosinophils for the EOSINOT03 cDNA library were isolated from 200 to 400 ml of peripheral blood drawn from six allergic, asthmatic donors, five of whom were male. The donors ranged in age from 31 to 42 and were allergic to common allergens such as dust, mold, pollen, animals, and, in one case, penicillin and some foods. Donors were medication-free for at least one week prior to blood donation. The whole blood samples were diluted 1:2 with cold 1×PIPES/glucose buffer, and each 25 ml of blood was layered onto a 15 ml cushion of 60% PERCOLL (Sigma Aldrich, St. Louis Mo.; 144 ml PERCOLL/72 ml H₂O/24 ml 10×PIPES) in a 50 ml conical tube (on ice). The tubes were centrifuged at 2000 rpm at 4 C. for 35 minutes to separate mononuclear cells from granulocytes and red blood cells (RBCs). The serum, mononuclear cells, and PERCOLL were aspirated, and the sides of the each tube were wiped with a cotton swab. Following hypotonic shock and differential centrifugation to remove RBC ghosts, eosinophils were separated from neutrophils by negative selection with magnetic anti-CD16 beads (Miltenyi Biotechnology, Auburn Calif.). All eosinophil preparations were greater than 98% pure.

Pooled eosinophils were centrifuged at 1000 rpm at room temperature for 5 minutes and then placed on ice. The supernatant was removed, and the cell pellet was washed once with phosphate buffered saline and dissolved in 5 ml of buffer consisting of 5 M guanidine isothiocyanate, 10 mM EDTA, 50 mM Tris-HCl (pH 7.5) and 8% 1-mercaptoethanol. A five-fold volume of 4M LiCl was added to the buffer, and the mixture was stored in the refrigerator for more than sixteen hours. After centrifugation, the precipitate was washed once with 3M LiCl and centrifuged once more. The second precipitate was dissolved in a solution of 0.1 % sodium dodecyl sulfate, 1 mM EDTA, and 10 mM Tris-HCl (pH 7.5). The suspension was frozen at −70 C. and then vortexed during thawing.

Total RNA was extracted twice with phenol chloroform, once with chloroform, and precipitated with ethanol. Following centrifugation, the RNA pellet was redissolved in DEPC-treated, distilled deionized water (DEPC-ddH₂O) and pelleted through a CsCl gradient. The RNA was extracted with acid phenol (1×, pH 4.0, Ambion, Austin Tex.), precipitated with ethanol and resuspended in DEPC-ddH₂O. The RNA was treated with RNAse-free DNAse (Epicentre Technologies, Madison Wis.) for 15 minutes, extracted with chloroform, precipitated, washed with ethanol, and dissolved in DEPC-ddH₂O.

The mRNA was handled according to the recommended protocols in the SUPERSCRIPT plasmid system (Life Technologies). cDNA synthesis was initiated with a Not I-oligo d(T) primer. Double stranded cDNA was blunted, ligated to Sal I adaptors, digested with Not I, fractionated on a SEPHAROSE CL4B column (APB), and those cDNAs exceeding 400 bp were ligated into the Not I and Sal I sites of the pSPORT vector (Life Technologies).

II Isolation and Sequencing of cDNA Clones

Plasmid DNA was released from the cells and purified using the REAL Prep 96 plasmid kit (Qiagen, Valencia Calif.). This kit enabled the simultaneous purification of 96 samples in a 96-well block using multi-channel reagent dispensers. The recommended protocol was employed except for the following changes: 1) the bacteria were cultured in 1 ml of sterile Terrific Broth (BD Biosciences, San Jose Calif.) with carbenicillin at 25 mg/L and glycerol at 0.4% for 19 hours; 2) the cells were lysed with 0.3 ml of lysis buffer; and 3) the plasmid DNA pellet was precipitated using isopropanol and then resuspended in 0.1 ml of distilled water. After the last step in the protocol, samples were transferred to a 96-well block for storage at 4 C.

The cDNAs were prepared for sequencing using the MICROLAB 2200 system (Hamilton) in combination with the DNA ENGINE thermal cyclers (MJ Research). The cDNAs were sequenced by the method of Sanger and Coulson (1975; J Mol Biol 94:441-448) using a PRISM 373 or 377 sequencing system (ABI). Most of the isolates were sequenced according to standard ABI protocols and kits with solution volumes of 0.25×-1.0×concentrations or using solutions and dyes from APB.

III Identification and Completion of the Full Length cDNA Encoding EGPCR

EGPCR was identified in Incyte clone 429905 from the EOSINOT03 cDNA library. The cDNA of Incyte clone 429905 was sequenced by in vitro transposon insertion using the Primer Island Transposon kit (ABI). The cDNA clone containing the ˜3.5 kb insert was mixed and incubated with transposition reagents which contained the artificial AT2 transposon provided in the kit for one hour at 30 C. The DNA mixture was electroporated into DH10OB competent E. coli cells (Life Technologies) and plated onto LB agar plates containing ampicillin for plasmid selection and trimethoprim/chloramphenicol for transposon selection. Resistant colonies were mapped for transposon insertion site by PCR analysis, and DNA of clones with transposon insertion at various locations throughout the insert were sequenced as described above. The nucleic acid sequence was assembled using the Autoassembler program (ABI) and edited manually. When the complete sequence was compiled from data utilizing both DNA strands, the reading frame was determined.

IV Northern Analysis

Membrane-based northern analysis was performed on 2 μg of poly A+ RNA samples from a variety of human tissues on the multiple tissue northern blots (MTN, Clontech, Palo Alto Calif.). Two DNA fragments were isolated from the Incyte clone 402295, a 539 bp Xba I-BamH I fragment and a 984 bp BamH I-EcoR I fragment. The fragments corresponded to most of the coding region between amino acid residues 173 and 353. The fragments were labeled with [³³P] isotope using the random primer labeling method (High Primer DNA labeling kit; Boehringer-Mannheim, Indianapolis Ind.). Hybridization of the probe to the MTN blot was under conditions of high stringency (5×SSC, 50 mM NaPO₄, pH 7.4, 1×Denhardts, 2% SDS and 100 μg/ml salmon sperm DNA) at 65 C. for more than sixteen hours. The blots were washed 1) two to three with 2×SSC at room temperature times, and 2) one to two times with 0.2×SSC, 0.1% SDS at 50 C., and 3) autoradiographed at −80 C.

V Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST2 to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin et al. ((1988) Proc Natl Acad Sci 85:841-845), BLAST matches between a query sequence and a database sequence were evaluated statistically and only reported when they satisfied the threshold of 10⁻²⁵ for nucleotides and 10⁻⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the stringency for an exact match was set from a lower limit of about 40 (with 1-2% error due to uncalled bases) to a 100% match of about 70.

The BLAST software suite (NCBI, Bethesda Md.), includes various sequence analysis programs including “blastn” that is used to align nucleotide sequences and BLAST2 that is used for direct pairwise comparison of either nucleotide or amino acid sequences. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity is measured over the entire length of a sequence. Brenner et al. (1998; Proc Natl Acad Sci 95:6073-6078, incorporated herein by reference) analyzed BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another, and those having local similarity of at least 82% were combined and reassembled. Bins having templates with less than 95% local identity were split. Templates were subjected to analysis by STITCHER/EXON MAPPER algorithms that determine the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. Nos. 08/812,290 and 08/811,758, both filed Mar. 6, 1997; in 08/947,845, filed Oct. 9, 1997; and in 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.). The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

VI Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding EGPCR that have been mapped result in the assignment of all related regulatory and coding sequences to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VII Hybridization Technologies and Analyses

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0. 1% SDS and acetone, etching in 4% hydrofluoric acid (VWR Scientific Products, West Chester Pa.), coating with 0.05% aminopropyl silane (Sigma Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60 C.; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-microcolumn (APB). The purified probe is heated to 100° C. for five min, snap cooled for two min on ice, and used in membrane-based hybridizations as described below.

Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNase inhibitor, 1 μl reverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C. for two hr. The reaction mixture is then incubated for 20 min at 85 C., and probes are purified using two successive CHROMA SPIN+TE 30 columns (Clontech). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65 C. for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexese, XOMAT-AR film (Eastman Kodak, Rochester N.Y.) is exposed to the membrane overnight at −70 C., developed, and examined visually.

Polymer Coated Slide-based Hybridization

Probe is heated to 65 C. for five min, centrifuged five min at 9400 rpm in a 5415 C. microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C. in 1×SSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20×microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

VIII Transcript Imaging

A transcript image was performed using the LIFESEQ GOLD database (Sep01release, Incyte Genomics). This process allowed assessment of the relative abundance of the expressed polynucleotides, an estimate of the expressed protein, in all of the cDNA libraries. Criteria for transcript imaging can be selected from category, number of cDNAs per library, library description, disease indication, clinical relevance of sample, and the like.

All sequences and cDNA libraries in the LIFESEQ database have been categorized by system, organ/tissue and cell type. For each category, the number of libraries in which the sequence was expressed were counted and shown over the total number of libraries in that category. In some transcript images, all normalized or pooled libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be disregarded or removed where clinical relevance is emphasized. Conversely, fetal tissue may be emphasized wherever elucidation of inherited disorders or differentiation of particular cells or organs from stem cells (such as nerves, heart or kidney) would be furthered by removing clinical samples from the analysis.

Transcript imaging can also be used to support data from other methodologies such as northern and microarray analyses. Two tissue-specific transcript images, lung and peripheral blood, for Incyte clone 429905 are shown below. The first column shows library name; the second column, the number of cDNAs sequenced in that library; the third column, the description of the library; the fourth column, absolute abundance of the transcript in the library; and the fifth column, percentage abundance of the transcript among the cDNAs in the library.

Category: Respiratory (Lung)

| | | Description | | |
|---|---|---|---|---|
| Library* | cDNAs | of Tissue | Abundance | % Abundance |
| LUNPTMC01 | 3046 | lung, aw/mets | 1 | 0.0328 |
| | | leiomyoSAR to | | |
| | | lung, 58F, lg | | |
| | | cDNA | | |
| LUNGNOT28 | 3851 | lung, | 1 | 0.0260 |
| | | mw/adenoCA, | | |
| | | 53M, m/ | | |
| | | LUNGTUT17 | | |

*cDNA libraries with less than 100 cDNAs, fetal, mixed, subtracted, and normalized libraries were removed from this analysis.
**aw/ = associated with: mw/ = matched with

When used in a tissue-specific manner with lung tissue, the expression of SEQ ID NOs:1 and 2 are diagnostic of lung cancer. Expression was not found in normal LUNPTMC01 tissue matched with LUNPTMC01(above); in normal lung tissue (LUNGNOE02, LUNGNOM01, LUNGNOT01, LUNGNOT02, LUNGNOT03, LUNGNOT04, LUNGNOT12, LUNGNOT14, LUNGNOT18, LUNGNOT20, LUNGNOT22, LUNGNOT23, LUNGNOT25, LUNGNOT27, LUNGNOT31, LUNGNOT33, LUNGNOT35, LUNGNOT37, LUNGNOT40, LUNGTMT03, and LUNGTMT04); in lung tissue from subjects diagnosed with idiopathic pulmonary disease (LUNGDIN02 or LUNGDIS03), in lung tissue from asthmatics (LUNGAST01 LUNGNOT33 LUNGTMT38, and LUNGTMT39); in lung tissue from a subject diagnosed with emphysema (LUNGNOT20 ); in lung tissue from a subject diagnosed with pneumonitis, (LUNGNOT15); in lung tissue from subjects diagnosed with squamous cell CA (LUNGTUE01, LUNGTUP11, LUNGTUT03, LUNGTUT07, LUNGTUT09, LUNGTUT11, LUNLTUE01, LUNLTUE02, LUNLTUT02, LUNLTUT04, and LUNLTUT11); and in lung tissue from subjects diagnosed with large cell CA (LUNGTUP12 and LUNGTUP13).

Category: Hematopoietic/Immune

| | | Description | | |
|---|---|---|---|---|
| Library* | cDNAs | of Tissue | Abundance | % Abundance |
| EOSITXT01 | 8975 | periph blood, | 7 | 0.0780 |
| | | eosinophils, | | |
| | | t/IL-5 | | |
| EOSIHET02 | 9259 | periph blood, | 5 | 0.0540 |
| | | eosinophils, | | |
| | | hypereosinophil- | | |
| | | ia, 48M | | |
| EOSINOT02 | 2356 | periph blood, | 1 | 0.0424 |
| | | eosinophils, | | |
| | | asthma, M/F | | |
| EOSINOT03 | 3723 | periph blood, | 1 | 0.0269 |
| | | eosinophils, | | |
| | | asthma, M/F | | |

*cDNA libraries treated with more than one activating substance, fetal, mixed, subtracted, and normalized libraries were removed from this analysis.

When used with a peripheral blood sample, the expression of SEQ ID NOs:1 and 2 are diagnostic of immunological response best characterized as eosinophilia. In peripheral blood, expression was not found in eosinophils collected from nonallergic subjects (EOSINOT01), in dendritic cells (DENDNOT01 and DENDTNT01), in blast cells (AMLBNOT01), or in lymphocytes (TLYMNOT01, TLYMNOT02, TLYMUNT03, TLYMNOR01, TLYMNOT01, TLYMNOT02, TMLR2DT01 and TMLR3DT02).

IX Complementary Molecules

Molecules complementary to the cDNA, from about 5 (PNA) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

X Expression of Recombinant EGPCR Protein

Expression of EGPCR was demonstrated by subcloning the cDNAs with or without an amino terminal Myc-tag into appropriate vectors and introducing the constructs into mammalian host cells, for example, HEK 293. A 539 bp Xba I—BamH I fragment and a 984 bp BamH I—EcoR I fragment of SEQ ID NO:2 containing the entire coding region was subcloned into a bi-cistronic green fluorescent protein (GFP) expression vector (CIEN.A12, Pfizer, Groton Conn.). The vector links the expression of the inserted cDNA with that of GFP via an internal ribosomal entry site (IRES). Although the inserted cDNA and GFP were co-transcribed as a single messenger RNA molecule, they were translated separately. The expression of GFP indicated transcription and translation of the inserted cDNA.

A Myc tag was engineered in front of the amino terminus of EGPCR using PCR methods well known in the art. Primers containing the Myc tag sequence joined with sequence encoding EGPCR and were used to detect EGPCR expression in transfected cells. The resulting expression constructs were transfected into HEK 293 cells (Kelly Mayo, Department of Biochemistry, Northwest University, Chicago Ill.) using LipofectAMINE reagent (Life Technologies). Stable cell lines were obtained by co-transfection with a vector containing a hygromycin resistance gene and subsequent culture in the presence of 5 μg/ml hygromycin. Transfected cells expressing GFP were further enriched by FACS sorting.

XI Production of EGPCR Specific Antibodies

An oligopeptide of 10 amino acid residues (equivalent to residues 121 to 130 of full-length EGPCR) located within the extracellular region of EGPCR was synthesized using methods well-known in the art. Two rabbits were immunized with the oligopeptide-KLH complex in complete Freund's adjuvant (Charles River, Wilmington Mass.) using methods well-known in the art. The resulting antisera, R5833, was tested for antipeptide activity by ELISA. R5833 antiserum recognized recombinant protein expressed in mammalian HEK 293 cells by Western blot analysis.

XII Western Blot Analyses and Deglycosylation

Transfected HEK 293 cells containing the EGPCR expression constructs (429905-GFP and Myc-429905-GFP) were lysed with buffer containing 25 mM Tris-HCl, pH 7.4, 1 mM MgCl₂, 100 mM NaCl, 2 mM EDTA, 1% Triton X-100, 4 mM Pefabloc (Boehringer Mannheim) and sonicated with microtips (70% duty power) using two to three pulses. Proteins were separated on a denaturing polyacrylamide gel (NUPAGE gels, Novex, San Diego Calif.) and transferred onto a nitrocellulose membrane as described in Ausubel (1997, Chap 11). After the blot was probed with antiserum R5833 in buffer and washed, the blot was incubated with a secondary antibody, HRP (horseradish peroxidase)-conjugated donkey anti-rabbit Ig, and visualized using ECL (enhanced chemiluminescence) system (APB).

Total cell membranes were isolated by suspension of cell pellets in ice-cold sucrose buffer (0.25 M sucrose, 0.1 M KPO4, pH 7.4, 0.1 mM EDTA) and 4 mM Pefabloc, sonicated two to three times with microtips (70% duty power), and centrifuged at 100,000×g for 1 hour at 4 C. The S100 supernatant fraction was transferred to a fresh tube, the residual pellet was homogenized in ice cold “PEG” buffer (0.1 M KPO₄, pH 7.4, 0.1 mM EDTA, 20% glycerol). Deglycosylation was conducted using N-glycosidase F deglycosylation kit (Boehringer-Mannheim) following the manufacturer's instructions. The glycoprotein samples were heated at 95° C. for three minutes in denaturation buffer with 1% β-mercaptoethanol (v/v), reconstituted in buffer containing N-glycosidase, and incubated for 1 hour at 37° C. The reaction was stopped by addition of SDS-sample buffer and heating at 95° C. for three minutes.

XIII Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffmity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding With the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories, Palo Alto Calif.), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30 C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl-β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

XVI Immunopurification Using Antibodies

Naturally occurring or recombinantly produced protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the purified protein is collected.

XVII Antibody Arrays

Protein:Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt et al. (2000) Nat Biotechnol 18:989-94).

XVIII Demonstration of EGPCR Expression in Cells

Expression of EGPCR on the surface of transfected HEK 293 cells was demonstrated by immunofluorescent antibody staining followed by flow cytometry. Briefly, a Myc-tag was fused to the amino terminus of EGPCR immediately after the signal peptide in the bi-cistronic expression vector. The Myc-tagged EF-GFP cDNA construct (Myc429905-GFP) or the control vector (GFP Control) was transfected into HEK 293 cells. Cells were stained first with murine anti-Myc antibody (Clone 9E10, Boehringer-Mannheim) or control murine immunoglobulin (PharMingen, San Diego Calif.), and second with phycoerythrin (PE)-conjugated anti-mouse Ig (PharMingen). The cells were analyzed for GFP fluorescence intensity and phycoerythrin intensity on a FACSort flow cytometer (Becton Dickinson, San Diego Calif.). Anti-Myc antibody stained cells transfected with Myc-tagged EF-GFP cDNA construct (Myc-429905-GFP) and failed to stain cells transfected with control vector. Cell surface expression was also demonstrated by fluorescence microscopy of HEK 293 transfectants expressing Myc-tagged EGPCR.

XIX Flow Cytometry and Immunofluorescence Staining

Cells were harvested and stained with a mouse anti-Myc antibody (Clone 9E10, Boehringer Mannheim) or isotype matched control mouse IgG (Pharmingen) at 4 C. for 30 minutes, washed, and then incubated with phycoerythrin (PE)-conjugated anti-mouse Ig (Pharmingen). The cells were analyzed for GFP fluorescence intensity and phycoerythrin intensity on a FACSORT flow cytometer (Becton Dickinson). Transfected cells were maintained in tissue culture flasks and incubated with primary and secondary antibodies at 37 C. for 30 minutes and washed as described above. Immunofluorescence of the cells was visualized immediately with an epifluorescent microscope (Nikon, Tokyo, Japan).

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A purified protein comprising an amino acid sequence selected from:
a) a protein having an amino acid sequence of SEQ ID NO:1;
b) a protein with 90% identity to the amino acid sequence of SEQ ID NO:1 which binds R5844 antiserum;
c) an antigenic determinant of the protein consisting of residues 121 to 130 of SEQ ID NO:1; and
d) a biologically active portion of the protein selected from residues 1-350, 173-353, and 332-657 of SEQ ID NO:1.

2. A composition comprising the protein of claim 1 and a pharmaceutical carrier.

3. A method for using a protein to screen a plurality of molecules or compounds to identify at least one ligand, the method comprising:
a) combining the protein of claim 1 with the molecules or compounds under conditions to allow specific binding; and
b) detecting specific binding, thereby identifying a ligand which specifically binds the protein.

4. The method of claim 3 wherein the molecules or compounds are selected from DNA molecules, RNA molecules, peptide nucleic acids, peptides, proteins, mimetics, agonists, antagonists, inhibitors, and drugs.

5. A method of using a polypeptide to purify a molecule or compound which specifically binds the polypeptide, the method comprising:
a) combining the polypeptide of claim 1 with a sample under conditions to allow specific binding;
b) recovering the bound-polypeptide; and
c) separating the polypeptide from the molecule or compound, thereby obtaining purified molecule or compound.

6. An array containing the protein of claim 1.

7. A method of using a protein to prepare and purify a polyclonal antibody comprising:
a) immunizing a animal with a protein of claim 1 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified polyclonal antibodies.

8. A method of using a protein to prepare a monoclonal antibody comprising:
a) immunizing a animal with a protein of claim 1 under conditions to elicit an antibody response;
b) isolating antibody producing cells from the animal;
c) fusing the antibody producing cells with immortalized cells in culture to form monoclonal antibody producing hybridoma cells;
d) culturing the hybridoma cells; and
e) isolating from culture monoclonal antibodies which specifically bind the protein.

9. A method for using a protein to screen a plurality of antibodies to identify an antibody which specifically binds the protein, the method comprising:
a) contacting a plurality of antibodies with the protein of claim launder conditions to form an antibody:protein complex, and
b) dissociating the antibody from the antibody:protein complex, thereby obtaining an antibody which specifically binds the protein.

10. A method for testing a molecule or compound for effectiveness as an agonist, the method comprising:
a) exposing a sample comprising a protein of claim 1 to a molecule or compound, and
b) detecting agonist activity in the sample.

11. A method for testing a molecule or compound for effectiveness as an antagonist, the method comprising:
a) exposing a sample comprising a protein of claim 1 to a molecule or compound, and
b) detecting antagonist activity in the sample.

12. An antibody which specifically binds the protein of claim 1

13. A method for using an antibody to detect expression of a protein in a sample, the method comprising
a) combining the antibody of claim 12 with a sample under conditions which allow the formation of antibody:protein complexes; and
b) detecting complex formation, wherein complex formation indicates expression of the protein in the sample.

14. The method of claim 13 wherein complex formation is compared with standards and is diagnostic of lung cancer.

15. The method of claim 13 wherein complex formation is compared with standards and is diagnostic of eosinophilia.

16. A composition comprising an antibody of claim 12 and a labeling moiety.

17. A method for using an antibody to immunopurify a protein, the method comprising:
a) attaching an antibody of claim 12 to a substrate,
b) exposing the antibody to a sample containing protein under conditions to allow antibody:protein complexes to form,
c) dissociating the protein from the complex, and
d) collecting the purified protein.

18. An array containing the antibody of claim 12.
